(19) United States
(12) Patent Application Publication
(10) Pub. No.: US 2012/0144903 A1
(43) Pub. Date: Jun. 14, 2012

(54) METHOD OF EXTRACTING CHLOROPHYLL AND MEASURING CONSISTENCY OF CHLOROPHYLL

(75) Inventor: Chungpin Liao, Taichung (TW)

(73) Assignee: INNOT BIOENERGY HOLDING CO., George Town (KY)

(21) Appl. No.: 13/076,268

(22) Filed: Mar. 30, 2011

(30) Foreign Application Priority Data
Dec. 13, 2010 (CN) 201010585266.9

(51) Int. Cl. G01N 21/25; C07D 487/22

(52) U.S. Cl. 73/61.48; 540/145

(57) ABSTRACT

An exemplary method of extracting chlorophyll is provided according to the embodiments of the present invention. The method includes the following steps: providing a plant leaf containing chlorophyll therein and smashing the plant leaf; extracting the chlorophyll from the smashed plant leaf by using an organic solvent, thereby achieving an extracted solution containing chlorophyll therein; filtering the extracted solution and concentrating the filtered solution; and collecting the concentrated solution containing chlorophyll. An exemplary method of measuring a consistency of the chlorophyll is also provided in the present invention.

### CROSS-REFERENCE TO RELATED APPLICATION

This application claims priority of Chinese Patent Application No. 201010585266.9, filed on Dec. 13, 2010, entitled “Method Of Extracting Chlorophyll And Measuring Consistency Of Chlorophyll” by Chungpin Liao, the disclosure of which is incorporated herein by reference in its entirety.

### FIELD OF THE INVENTION

The present invention relates to a material chlorophyll, and more particularly to a method of extracting chlorophyll and a method of measuring consistency of chlorophyll.

### BACKGROUND OF THE INVENTION

Chlorophyll is a kind of major material involved in photosynthesis process. There are generally several kinds of chlorophylls, for example, chlorophyll a, chlorophyll b, chlorophyll c, chlorophyll d, and chlorophyll e. The chlorophyll a and the chlorophyll b normally exist in leaves of senior plants. The chlorophylls are usually insoluble in water, making it difficult to extract the chlorophylls from the senior plants.

### SUMMARY OF THE INVENTION

The present invention provides a method of extracting chlorophyll and a method of measuring consistency of chlorophyll that solves the problems encountered with conventional ways for extracting chlorophyll and measuring consistency of chlorophyll. The advantages of the present invention will be understood more readily after a consideration of the drawings and the detailed description of the preferred embodiments.

### BRIEF DESCRIPTION OF THE DRAWINGS

The present invention will become more fully understood from the detailed description given herein below for illustration only, and thus are not limitative of the present invention, and wherein:

FIG. 1 shows several kinds of chlorophylls in the natural world;

FIG. 2 shows a process of preserving a plant leaf according to one embodiment of present invention;

FIG. 3 shows a flow chart illustrating a process of extracting chlorophyll from the plant leaf according to one embodiment of present invention;

FIG. 4 shows comparison diagrams of absorption spectrums of three organic solvents; and

FIG. 5 shows consistency of the chlorophylls of three organic solvents.

### DETAILED DESCRIPTION OF THE INVENTION

Reference will now be made to the drawings to describe an exemplary embodiment in detail.

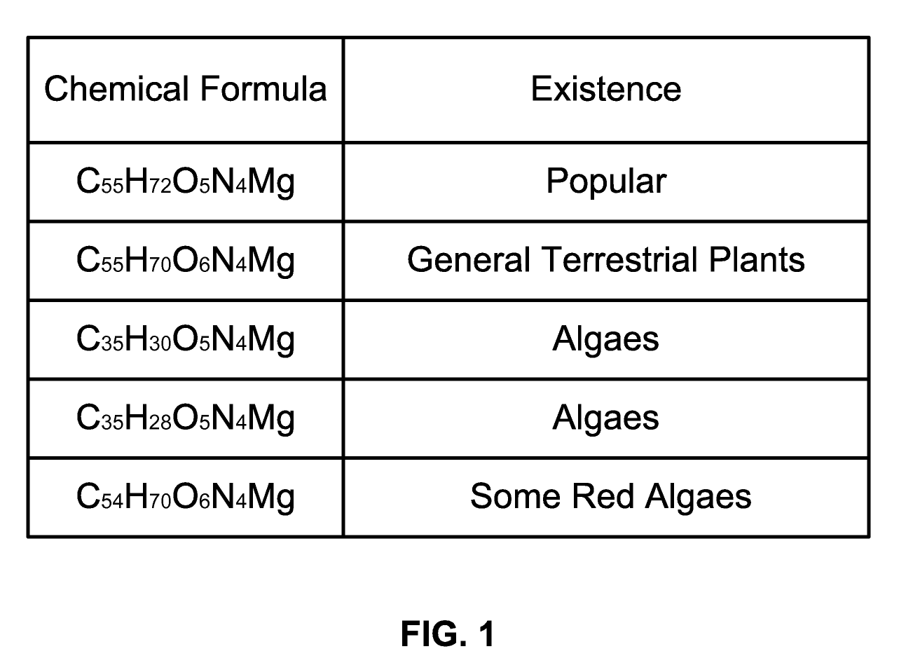

FIG. 1 shows several kinds of chlorophylls in the natural world. The chlorophylls are important pigments for photosynthesis. In the illustrated embodiment, a plant leaf of a terrestrial plant is chosen as a source for extracting chlorophyll therefrom.

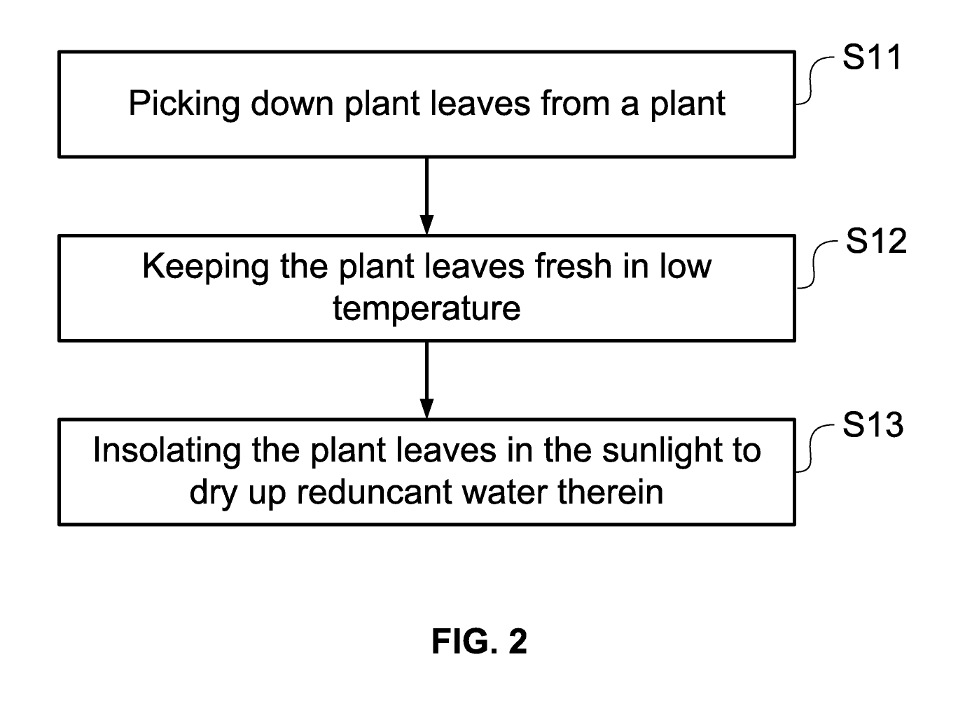

Referring to FIG. 2, it shows a process of preserving plant leaves according to one embodiment of the present invention. The plant leaves hereinafter refer to a principal body of the plant leaves. The processing of preserving the plant leaf includes the following steps: step S11, picking down the plant leaf from a plant; step S12, keeping the plant leaf fresh in a low temperature; and step S13, insolating the plant leaf in the sunlight to dry up redundant water therein. That is, the plant leaf is kept fresh in a low temperature after it is picked down from a plant, and is insolated in the sunlight to dry up redundant water therein.

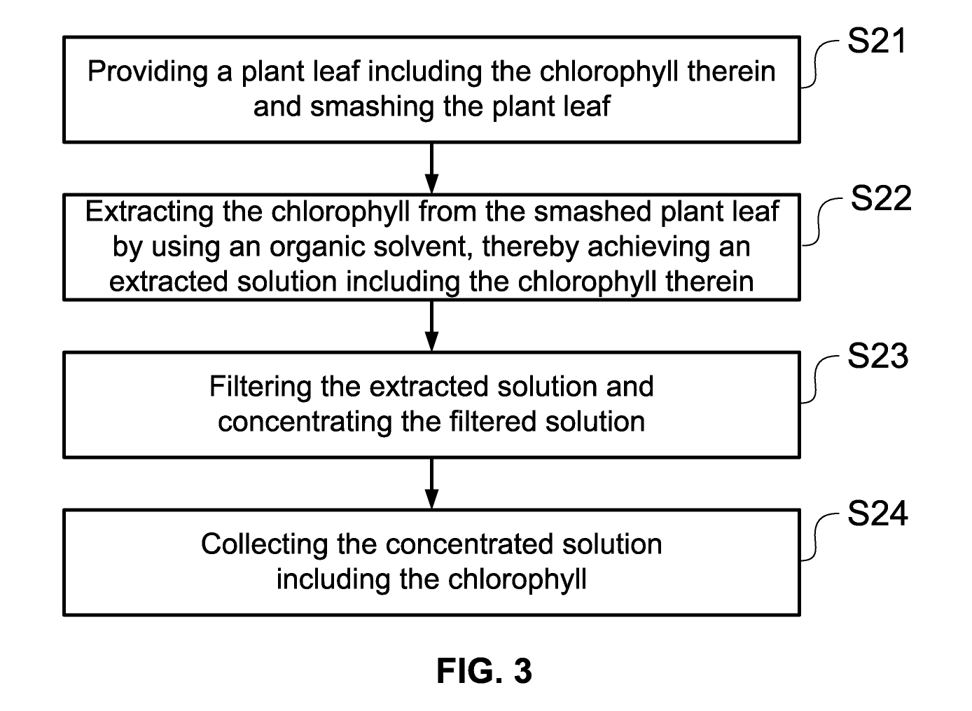

FIG. 3 shows a flow chart illustrating a process of extracting chlorophyll from the plant leaf according to one embodiment of the present invention. The process includes the follows steps:

step S21: providing a plant leaf containing chlorophyll therein and smashing the plant leaf; The plant leaf containing one or more kinds of chlorophylls therein is provided and is further smashed into powder. The plant leaf is pre-processed as shown in FIG. 2 and is further soaked into liquid nitrogen. The soaked plant leaf is then smashed into powder using a disintegrator (not shown), and a diameter of the powder is about 0.7 millimeters. The powder is preferably stored under a dark condition with a low temperature. Preferably, liquid nitrogen is mixed with the plant leaf in the smashing procedure due to an influence of temperature.

step S22: extracting the chlorophyll from the smashed plant leaf by using an organic solvent, thereby achieving an extracted solution containing chlorophyll therein; The powder is mixed with one or more solutions, and the chlorophyll in the powder is extracted using a centrifuge. The solutions are selected from the groups consisting of ethyl alcohol (C₂H₅OH), acetone, dimethyl formamide (DMF), and dimethyl sulfoxide (DMSO). The extraction procedure is preferably performed by using a disintegrator in a dark condition.
The ethyl alcohol and the acetone can be conveniently applied in the extraction procedure due to good volatilizations. Additional ventilation equipments are needed when the DMF and the DMSO are applied in the extraction procedure due to their harm to human health. The additional ventilation equipments include a vacuum drying oven and an air exhaust device.

step S23: filtering the extracted solution and concentrating the filtered solution; The powder of the plant leaf in the extracted solution is filtered to remove unwanted particles by one or more pieces of filter paper. The filter paper may be selected from a group consisting of qualitative filter paper, quantitative filter paper, and fiber glass filter paper. The filtering procedure of the extracted solution is preferably to be completed within 24 hours.
The filtered solution is then concentrated, and a thickener may be used in the concentration procedure.

step S24: collecting the concentrated solution containing the chlorophyll; The concentrated solution may be stored in a reefer chamber, and a preferred condition temperature in the reefer chamber is 4 degree Celsius. The concentrated solution should be used to measure characteristic of the chlorophyll a and the chlorophyll b as soon as possible. Otherwise, the concentrated solution should be stored in a dark condition having a temperature of −20 degree Celsius for no more than 14 days, or −70 degree Celsius for no more than three months.

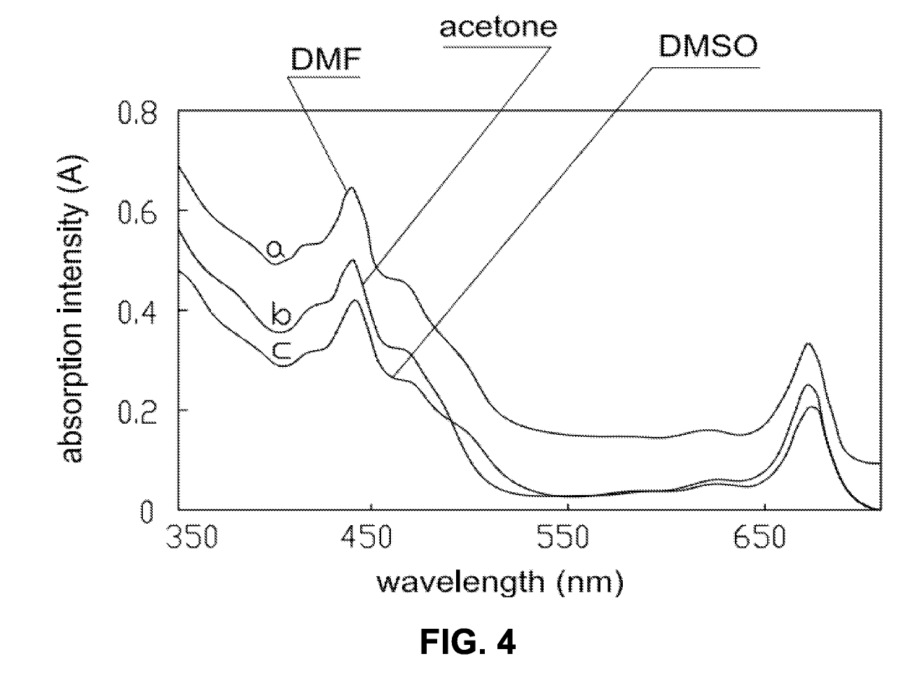

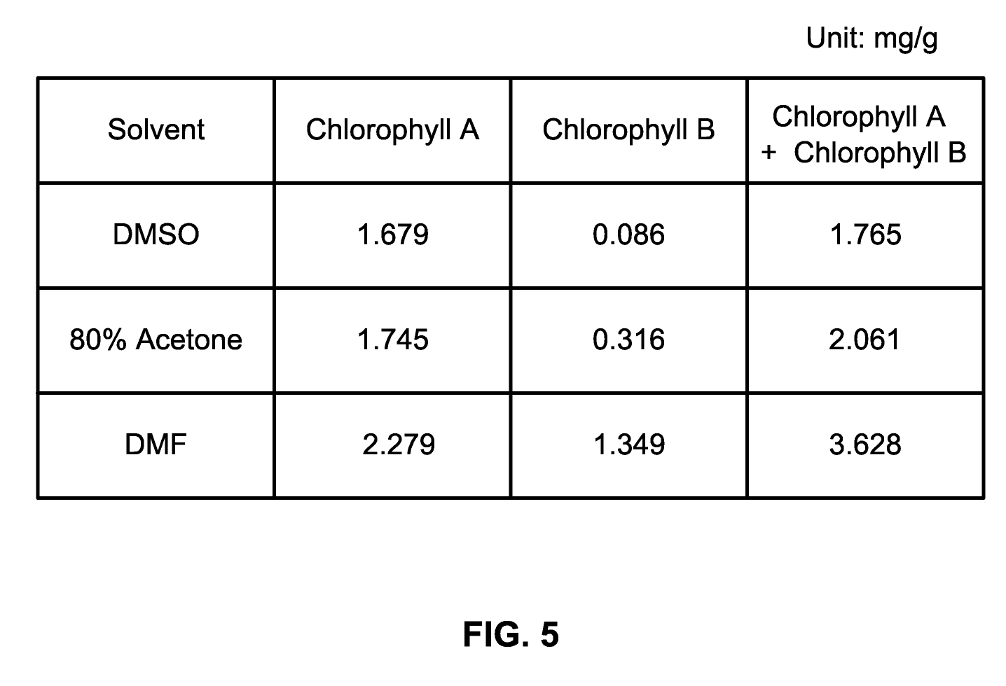

A visible spectrophotometer may be used to measure an absorption intensity of the chlorophyll a and the chlorophyll b in different solutions. Referring to FIG. 4 and FIG. 5, FIG. 4 shows comparison diagrams of absorption spectrums of three solutions, and FIG. 5 shows the consistency of the chlorophyll of the solutions, whereby the line labeled “a” represents the DMF solution, the line labeled “b” represents the acetone solution, and the line labeled “c” represents the DMSO solution. As shown in FIG. 4 and FIG. 5, as far as the chlorophyll from a spinach is concerned, a peak value of absorption of the chlorophyll a is around 662 nanometers, and a peak value of absorption of the chlorophyll b is around 455 nanometers. Therefore, the consistency of the chlorophyll a, the chlorophyll b, and the total consistency of the chlorophyll a and the chlorophyll b may be calculated by the peak value with known formulas. Consequently, the consistency of the chlorophyll extracted from the DMF solution is maximal, and an extracting ratio of the chlorophyll is correspondingly maximal.

The above-described exemplary embodiment may be applied as an evaluation process for extracting the chlorophyll to make a battery using the chlorophyll. When the battery is made, changes of the chlorophyll in the battery may be measured. For example, a value of PH/ORP (pondus hydrogenii/oxidation reduction potential) may be measured to detect whether any poisonous composition is generated during the use of the battery.

It is to be understood, however, that even though numerous characteristics and advantages of preferred and exemplary embodiments have been set forth in the foregoing description, together with details of the structures and functions of the embodiments, the disclosure is illustrative only; and that changes may be made in detail within the principles of present disclosure to the full extent indicated by the broadest general meaning of the terms in which the appended claims are expressed.

What is claimed is:

1. A method of extracting chlorophyll, comprising the steps of:
(i) providing plant leaves comprising the chlorophyll therein and smashing the plant leaves;
(ii) extracting the chlorophyll from the smashed plant leaves by using an organic solvent, thereby achieving an extracted solution comprising the chlorophyll therein;
(iii) filtering the extracted solution and concentrating the filtered solution; and
(iv) collecting the concentrated solution comprising the chlorophyll.

2. The method of claim 1, wherein the smashed plant leaves have an average diameter approximately 0.7 millimeters.

3. The method of claim 1, wherein the plant leaves are kept fresh by controlling a condition temperature before the smashing procedure, and are insolated in the sunlight to dry up redundant water therein.

4. The method of claim 1, wherein liquid nitrogen is mixed with the plant leaves in the smashing procedure of step (i).

5. The method of claim 1, wherein the organic solvent of step (ii) is selected from the group consisting of ethyl alcohol (C₂H₅OH), acetone, dimethyl formamide (DMF), and dimethyl sulfoxide (DMSO).

6. The method of claim 1, wherein the extraction procedure of step (ii) is performed by using a disintegrator in a dark condition.

7. The method of claim 1, wherein the extracted solution is filtered by one or more pieces of filter paper selected from qualitative filter paper, quantitative filter paper, and fiber glass filter paper.

8. The method of claim 1, wherein the filtering procedure of step (iii) is to be completed within 24 hours.

9. The method of claim 1, wherein the concentrated solution of step (iv) is stored in a reefer chamber.

10. The method of claim 9, wherein a condition temperature in the reefer chamber is approximately 4 degree Celsius.

11. A method of measuring consistency of chlorophyll, comprising the step of measuring a peak value of an absorption intensity of the chlorophyll in an extraction solution using a visible spectrophotometer.

12. The method of claim 11, wherein a consistency of the chlorophyll a, a consistency of the chlorophyll b, and a total consistency of the chlorophyll a and the chlorophyll b are calculated by their peak values.
